(19) United States
(12) Patent Application Publication
(10) Pub. No.: US 2005/0019321 A1
(43) Pub. Date: Jan. 27, 2005

(54) Method of obtaining protection against bystander allergen

(76) Inventors: Sarita Sehra, Indianapolis, IN (US); Johan Grooten, Lovendegem (BE)

Correspondence Address: TRASK BRITT, P.O. BOX 2550, SALT LAKE CITY, UT, 84110, US

(21) Appl. No.: 10/845,292

(22) Filed: May 13, 2004

Related U.S. Application Data
(63) Continuation of application No. PCT/EP02/12723, filed on Nov. 13, 2002.

(30) Foreign Application Priority Data
Nov. 13, 2001 (EP) 01204351.9

(51) Int. Cl. A61K 39/395

(52) U.S. Cl. 424/130.1; 424/131.1

(57) ABSTRACT

The present invention relates to a method of obtaining protection against, simultaneously, a specific allergen and a by-stander allergen. The method comprises the administration of a compound comprising an allergen binding domain and a Fc domain, wherein the bystander allergen is unrelated to the allergen bound by the allergen binding domain.

### CROSS-REFERENCE TO RELATED APPLICATION

This application is a continuation of co-pending PCT International Patent Application No. PCT/EP02/12723, filed on Nov. 13, 2002, designating the United States of America, and published, in English, as PCT International Publication No. WO 03/041731 A1 on May 22, 2003, the contents of which are incorporated by this reference.

### TECHNICAL FIELD

The present invention relates to a method to obtain protection against a bystander allergen, comprising the administration of a compound comprising an allergen binding domain and a Fc domain, whereby said bystander allergen is unrelated to the allergen bound by said allergen binding domain.

### BACKGROUND

Upon T-Cell Receptor (TCR)—ligation, ThO cells differentiate into distinct subsets characterized by their functions and cytokine production profiles (Mosmann and Coffman, 1989). Thus Th1 lymphocytes, characterized by the production of IL-2, IFN-γ and TNF-β contribute to cellular immunity whereas Th2 lymphocytes, mainly involved in humoral immunity, produce IL-4, IL-5 and IL-10. Numerous examples of the consequences on disease outcome of skewed Th1 to Th2 ratios have been reported. Polarized Th2 responses have been implicated in pathological situations, such as Leishmania major (Heinzel et al., 1991; Nabors et al., 1995), TBC (de Jong et al., 1997) human leprosy (Yamamura et al., 1991), and mycotic infections (Murphy et al., 1994). The contribution of Th1 cells relative to Th2 cells to the developing autoimmune response determines for a large part whether or not this response leads to clinical disease (Racke et al., 1994; Racke et al., 1995; Leonard et al., 1995). The chronic autoimmune graft-versus-host disease, which develops after the administration of mismatched lymphoid cells, can be prevented by switching a Th2 to a Th1 response through administration of IFN-γ at the time of cellular transfer (Donckier et al., 1994). Roussel et al. (1996) describe that the inefficiency of the immune response against a human glioma is caused by the presence of activated tumour-infiltrating lymphocytes, characterized by a predominant type 2 lymphokine production. These cytokines do not promote a tumoricidal immune response and therefore do not counteract the growth of the tumour.

In allergic asthma, also a predominant Th2 response has been noted-(Vogel, 1997). Asthma describes a heterogeneous collection of clinical symptoms such as reversible airway narrowing, airway hyper reactivity, and eosinophilic inflammation of the airways. Due to the chronic nature of asthma, structural and functional changes in the organ will occur on the long-term, resulting in airway remodeling and a further amplification of the syndrome. Clearly, sensitization to airborne environmental allergens, leading to atopy, is a major risk factor for asthma (reviewed in Holt et al., 1999). In sensitized individuals, exposure to the aeroallergen will trigger within minutes an acute response, resulting in airway constriction and difficult breathing. Interaction of the allergen with allergen-specific IgE antibodies bound to various effector cells through the Fcε receptor 1, provides the trigger for this acute reaction. The secreted inflammatory mediators in addition recruit eosinophils, mast cells, T lymphocytes and other circulating leukocytes to the site(s) of allergen challenge. Besides causing a recurrence of symptoms, this cellular infiltration by effector cells will persist upon chronic exposure to allergen, thus leading to chronic eosinophilic inflammation of the airways, characteristic for asthma (reviewed in Wills-Karp, 1999 and Galli, 2000).

Specific cytokines, various inflammatory mediators and allergen-specific IgE antibodies all contribute to the complex pathogenesis of asthma. However, increasing evidence indicates that Th2 cell-derived cytokines are pivotal in the generation and persistence of the disorder. Thus, IL-4 and IL-13 are critical in switching B-lymphocytes to produce of allergen-specific IgE. IL-3 controls the induction of mast cell proliferation and recruitment of lymphocytes, mast cells, and basophils. IL-5 is involved in growth and differentiation of eosinophils and B-lymphocytes, while IL-9 promotes growth and differentiation of mast cells. Finally, IL-10 inhibits IFN-γ production and classical activation of macrophages (reviewed in Corry and Kheradmand, 1999).

Accordingly, these Th2-derived cytokines, along with Ig E-mediated activities, represent important therapeutic targets, and strategies aimed at eliminating or neutralizing these activities are actively pursued by several researchers. These strategies involve the administration of neutralizing or antagonistic anti-cytokine or anti-IgE antibodies, administration of soluble cytokine receptors or peptido-mimetics of cytokine receptors. PCT International Patent Publication No. WO 9004979, for example, describes a method of preventing or reducing eosinophilia comprising administering an antagonist to human IL-5, such as a monoclonal antibody against IL-5. However, none of these methods is antigen specific. As a result, these methods will affect the targeted, allergic and local immune response as well as non-targeted, systemic immune responses or immune responses in other compartments of the body against pathogens and/or their antigens.

Alternative strategies for treatment of allergic diseases comprise selective suppression of the anti-allergen immune response. These approaches are all based on some form of active vaccination using injection of crude or purified allergen preparations and resulting in hyposensitization. Classically for this type of immunotherapy routes of administration are chosen that target systemic immune responses such as the subcutaneous route of administration. A more recent approach for immunotherapy, the so-called Saint-Remy technique (EP0178085 and EP0287361) uses autologous IgG antibodies complexed ex vitro to the relevant allergen(s). This approach generates fewer side effects due to the feasibility to apply smaller amounts of allergen. Hyposensitization has proven to be moderately effective in treating allergic diseases among which allergic rhinitis and asthma However, there are numerous difficulties with this form of treatment. Treatment schedules are cumbersome and prolonged courses of treatment are necessary, resulting in low patient compliance. Since the precise immune mechanism is not known, the cause of therapeutic failure usually cannot be established. Various improvements on the vaccination approach have been described to render hyposensitization more effective. These comprise among others encapsulation in or covalent attachment to liposomes of the allergen (U.S. Pat. No. 5,049,390), covalent attachment to the allergen of a saccharide (U.S. Pat. No. 5,073,628), and application of adjuvans that suppress formation of IgE antibodies and promote formation of IgM and IgG antibodies. Examples of the latter are a glycolipid extracted from maize tissue (U.S. Pat. No. 4,871,540) and preparations containing life or heat-killed mycobacteria such as Mycobacterium bovis Bacillus Calmette-Guerin or mycobacterial cell wall products (Azuma et al., 1976; Yang et al., 2000). All these methods as well as methods whereby the allergen is first modified by coupling to various bridging molecules such as antibodies and subsequently is administered to the recipient, as described for instance in PCT International Patent Publication No. WO9707218, have the important drawback that they encompass systemic administration of an allergen-containing composition to individuals that exhibit various degrees of atopy and/or anaphylaxis, and therefore are at risk of developing immediate hyperresponsiveness and/or anaphylactic shock in response to the treatment.

Yamauchi et al. (1983) describe that, in a model of allergic asthma, intravenous administration of specific IgG2 antibody prior to challenge with allergen inhibited the IgE induced bronchial response. The authors suggested that a direct competition between IgG2 and IgE for the antigen is responsible for the inhibition of the IgE induced bronchial response by blocking the trigger for the acute reaction. This treatment is a symptomatic treatment and will not affect allergic responses to secondary or bystander allergens.

Taken together, it is clear that the currently available methods to alleviate or cure asthma all have inherent limitations: a) antagonists or immune mediators such as anti-cytokine antibodies suppress non-related immune responses systemically, b) vaccination with allergen contains a risk of developing immediate hyperresponsiveness and/or anaphylactic shock, and c) intravenous (iv) administration of anti-allergen antibodies that compete with IgE's may immediately alleviate the symptoms without affecting the disease in the long run or without affecting allergic responses triggered by unrelated bystander antigens.

### DISCLOSURE OF THE INVENTION

Disclosed is a method for obtaining cross-protection against one or more bystander allergens, the method comprising: administering a compound comprising an allergen binding domain and a Fc domain, wherein the bystander allergens are unrelated to the allergen bound by the allergen binding domain.

Also disclosed is a pharmaceutical composition for the treatment of an allergic disease, the pharmaceutical composition comprising at least one compound comprising an allergen binding domain or allergen binding domains and a Fc domain, wherein the allergic disease is caused by at least one allergen other than the allergen or allergens bound by the allergen binding domain or allergen binding domains.

### BRIEF DESCRIPTION OF THE SEVERAL VIEWS OF THE DRAWINGS

FIG. 1A: Intranasal administration of anti-cat IgG2a decreases airway inflammation in h-cat sensitized mice. To determine the specificity of anti-cat IgG2a, mice were also treated with mismatched antibody (anti-OA IgG2a) followed by challenge with the correct allergen (h-cat). Thus h-cat sensitized and challenged mice were 1) treated with PBS (i.e., placebo) 2) treated with anti-cat IgG2a 3) treated with anti-OA IgG2a. Treatment was given on the first and second day of allergen challenge. 48 hrs after the second allergen challenge, total nucleated cell counts and differential cell counts were done on cytospin preparations of BAL fluid from these mice. Lymphocyte subsets CD4 and CD8 in BAL fluid were determined by flow cytometry. Both the total number of cells recovered by BAL and the number of each cell type are shown. Data are expressed as the mean±SEM of 5-7 mice per group. Significant decrease of total BAL cell count in anti-cat IgG2a treated group compared with PBS treated group (p<0.05). Similarly p<0.05 for BAL cell count of anti-OA IgG2a treated group and anti-cat IgG2a treated group.

FIG. 1B: Intranasal administration of anti-OA IgG2a to OA sensitized and challenged mice reduces cellular infiltration in BAL. To determine the specificity of anti-OA IgG2a, mice were also treated with mismatched antibody (anti-cat IgG2a) followed by challenge with the correct allergen (OA). Thus OA sensitized and challenged mice were 1) treated with PBS (i.e., placebo), 2) treated with anti-OA IgG2a 3) treated with anti-cat IgG2a. Treatment was given on the first and second day of allergen challenge. 48 hrs after the second allergen challenge, total nucleated cell counts and differential cell counts were done on cytospin preparations of BAL fluid from these mice. Lymphocyte subsets CD4 and CD8 in BAL fluid were determined by flow cytometry. Both the total number of cells recovered by BAL and the number of each cell type are shown. Data are expressed as the mean±SEM of 5-6 mice per group. Significant decrease of total BAL cell count and eosinophil cell count in anti-OA IgG2a treated group compared with PBS treated group (p<0.05). Similarly p<0.05 for total BAL cell and eosinophil cell count of anti-OA IgG2a treated group and anti-cat IgG2a treated group.

FIG. 1C: Administration of anti-OA IgG2a inhibits airway inflammation even after prolonged OA challenge by exposure to OA for consecutive days. Treatment of OA sensitized and challenged mice involved administration of 50 μg anti-OA IgG2a on day 0 and 3 of aerosol challenge or 100 μg of anti-OA IgG2a given on day 0 of aerosol challenge. 24 hrs after the last aerosol exposure, total cell counts and differential cell counts were done on cytospin preparations of BAL fluid from these mice. Both the total number of cells recovered by BAL and the number of each cell type are shown. Data represent the mean±SEM of 4-5 mice per group. Significant differences were observed between the total BAL cell count and eosinophil count of the anti-OA IgG2a treated group and the PBS treated group (p<0.05).

FIG. 1D: Intranasal instillation of anti-OA IgG (anti-OA IgG2a, anti-OA IgG2b or anti-OA IgG1) reduces airway inflammation in the OA sensitized mice, challenged by exposure to OA aerosol for 7 consecutive days. Mice were treated with 50μg of anti-OA IgG2a, anti-OA IgG2b, anti-OA IgG1 or PBS two hours before the first and fourth OA aerosol challenge. Twenty-four hours after the last aerosol exposure the total BAL cell count and differential cell counts in the cytospin preparations of BAL cells from these mice were determined. The total cells recovered by BAL are shown. Data represent the mean±SEM of 4-5 mice per group. Significant differences were observed between the treated and the placebo group (p<0.05).

FIG. 2: Inhibition of airway eosinophilia involves interaction with Fc receptors. Treatment involved intranasal administration of equimolar concentrations of F(ab′)₂ fragments of anti-OA IgG2a and native anti-OA IgG2a to OA sensitized mice on days 0 and 3 of a 7-day OA aerosol challenge. Twenty-four hrs after the last aerosol exposure the total BAL cell count and differential cell counts on cytospin preparations from these mice were determined. Data represent the mean±SEM of 5-7 mice per group. Significant differences in the BAL cell count and eosinophil count of the anti-OA IgG2a group and placebo group were observed (p<0.05). In contrast, the anti-OA F(ab′)₂ group did not differ from the placebo group.

FIG. 3: Histological analysis of the lungs. Instillation of anti-OA IgG but not of F(ab′)2 or placebo decreases the allergen induced eosinophilic airway inflammation (upper panel). Image A is representative for mice treated with IgG2a, image B for mice treated with F(ab′)2 or placebo. Lower panels present the number of airways free of mucus producing cells or containing over 50% PAS-positive cells. Lower image illustrates a typical PAS-staining. Mucus producing cells stain purple.

FIG. 4: Measurement of IFN-γ, IL-4 and IL-5 levels in supernatants of BAL fluid. Mice were treated according to the protocol of repetitive OA aerosol challenge and cytokine levels were determined by ELISA. Data represent the mean±SEM of 4-5 mice per group.

### DETAILED DESCRIPTION OF THE INVENTION

The present invention describes an approach for treatment of allergic diseases based on the conversion of the anti-allergen pathological response to a benign immune response. Indeed, surprisingly we found that a compound, comprising an allergen binding domain and a Fc domain can provide protection against the specific allergen while simultaneously providing cross protection against a bystander allergen which is unrelated to the allergen bound by said allergen binding domain. More specifically, we found that administration of the latter compound via the respiratory tract (i.e., nasal or intratracheal administration) resulted in cross protection against bystander allergen without affecting immune responses in other compartments of the body than the lungs. Moreover, the latter treatment results in long-term protection and disease modification and therefore does not only offer relief of the symptoms of the disease. The Fc domain of the compound of the present invention is the domain that binds specifically to the Fcγ receptor. Preferably, said compound is an anti-allergen antibody, said antibody being substantially free of allergen, more preferably an anti-allergen antibody of the IgG isotype, most preferably an IgG2a or IgG2b isotype.

Conversion of the anti-allergen pathogenic response implies a conversion of the polarized Th2 cell response, characteristic for allergic asthma, to a mixed Th1/Th2 response. Due to the pivotal role of Th2-cell derived cytokines in allergic asthma, this conversion reduces allergic asthma due to the diminished production of the causative Th2 cytokines and the mutual antagonistic activity of Th1 and Th2 cytokines. This approach does not involve a direct neutralization of immune mediators, thus leaving intact the functioning of the immune system, and has the advantage of antigen specificity so that it does not abrogate systemic beneficial Th2 responses against unrelated pathogens. In addition, this approach promises a substantial cure from asthma rather than a symptomatic treatment as a result of the correction of the fundamental cause of the disease, namely the anti-allergen Th2-polarized immune response. Furthermore, because the treatment does not require administration of allergen, either in its native form or modified, the disadvantages and health risks intrinsic to active vaccination strategies are avoided. Moreover, we found that it is unnecessary to treat the patient for several allergens, as a cross protection against unrelated bystander allergens is obtained.

It is a first aspect of the invention to provide a method to obtain cross protection against one or more bystander allergens, comprising the administration of a compound comprising an allergen binding domain and a Fc domain, whereby said bystander allergens are unrelated to the allergen bound by the allergen binding domain. Moreover, said cross protection is persistent in time and allows a cure of the allergic reaction, rather than being a symptomatic treatment.

A first embodiment is a method to obtain cross protection against one or more bystander allergens, whereby said compound is an anti-allergen antibody, comprising an Fc domain. Said antibody may be a natural occurring antibody, or an artificial protein wherein an antibody binding domain is linked to a Fc domain, possibly with the use of a linker region. Methods to make such artificial proteins are known to the person skilled in the art. A preferred embodiment is a method to obtain cross protection against one or more bystander allergens, whereby said antibody is an IgG isotype antibody, preferably an IgG2 isotype antibody.

It is another aspect of the invention to provide a pharmaceutical composition for the treatment of an allergic disease, comprising one or more compounds comprising an allergen binding domain and an Fc domain, whereby said allergic disease is caused by at least one allergen, other than the allergen or allergens bound by the allergen binding domain or allergen binding domains. Preferably, said compound is an anti-allergen antibody, substantially free from allergen. More preferably, said compound is an anti-allergen IgG type antibody, substantially free from other isotype antibodies and substantially free from allergen. Most preferably, said compound is an anti-allergen IgG2 type antibody, substantially free from other isotype antibodies and substantially free from allergen. Allergic diseases are known to the person skilled in the art and include, but are not limited to allergic asthma, allergic rhinitis, airway hyperreactivity and eosinophilic airway inflammation.

Still another aspect of the invention is the use of a compound comprising an allergen binding domain and an Fc domain for the manufacturing of a medicament for the treatment of an allergic disease. Preferably, said compound is an anti-allergen antibody, more preferably said antibody is directed against an antigenic structure of one of the causative agents. A preferred embodiment is the use of an IgG isotype according to the invention, whereby said disease is allergic asthma, allergic rhinitis, airway hyperreactivity and/or eosinophilic airway inflammation.

It is clear that the compounds of the present invention can be used as a medicament to treat allergic diseases such as asthma. It is thus a further embodiment of this invention to provide a method for the production of a pharmaceutical composition comprising the compounds of the present invention and mixing said compounds with a pharmaceutically acceptable carrier. The administration of said compounds or pharmaceutically acceptable salts thereof may be by way of nasal or inhaled administration. The active compound may be administered alone or preferably formulated as a pharmaceutical composition. A unit dose will normally contain 1 to 100 mg, for example 1 to 75 mg, 2 to 50 mg, 3 to 40 mg, 5 to 30 mg, 15 to 25 mg, 18 to 22 mg or 20 mg of compound or a pharmaceutically acceptable salt thereof. Unit doses will normally be administered once or more than once a day, for example 2, 3, or 4 times a day, or, once or more than once a month, for example 2, 3, 4, times a month, or once or more than once every 6 months, for example 2, 3, 4, times every six months, or, once or more than once a year, for example 2, 3, 4, 5, or 6 times a year. It is greatly preferred that the compound or a pharmaceutically acceptable salt thereof is administered in the form of a unit-dose composition, such as a unit dose nasal or inhaled composition. Such compositions are prepared by admixture and are suitably adapted for inhaled or nasal administration, and as such may be in the form of liquid preparations (nasal) or dry powders (inhalation). Preferably, compositions for inhalation are presented for administration to the respiratory tract as a snuff or an aerosol or solution for a nebulizer, or as a microfine powder for insufflation, alone or in combination with an inert carrier such as lactose. In such a case the particles of active compound suitably have diameters of less than 50 microns, preferably less than 10 microns, for example between 1 and 5 microns, such as between 2 and 5 microns. Dependant on the form of the unit-dose composition, devices suited for delivery typically are pressurized aerosols, nebulizers and dry powder inhalers designed for efficient and reproducible delivery, flexible dosing and allowing for patient control on intake of the composition. Where appropriate, small amounts of bronchodilators for example sympathomimetic amines such as isoprenaline, isoetharine, salbutamol, phenylephrine and ephedrine; xanthine derivatives such as theophylline and aminophylline and corticosteroids such as prednisolone and adrenal stimulants such as ACTH may be included. As is common practice, the compositions will usually be accompanied by written or printed directions for use in the medical treatment concerned.

The following definitions are set forth to illustrate and define the meaning and scope of various terms used to describe the invention herein.

IgG2 isotype antibody as used herein means an isotype antibody, derived either from a polyclonal or, preferentially, a monoclonal preparation and, if necessary, purified to a degree that it is free from immunological active amounts of antibodies of another isotype or of other immunological active compounds.

Substantially free of allergen means that the ratio of number of antibodies to the number of antibody-binding epitopes binding to said antibodies, as measured in vitro, before administration is at least 100/1, preferably 1000/1.

One or more IgG isotype antibodies, substantially free of other isotype antibodies means that the ratio of the total number of IgG isotype antibodies to the total number of non-IgG isotype antibodies, as determined in vitro, before administration is at least 100/1, preferably 1000/1.

A persistent cross protection is a protection whereby, after a contact with the bystander allergen, a significant decrease in inflammatory response is noticed, even after stopping the treatment for at least four days, preferably after stopping of the treatment for at least six days. The significance of the decrease may be evaluated by comparing the treatment, supposed to result in a persistent reduction of aeroallergen-induced inflammatory response with a placebo treatment.

Aeroallergens include, but are not limited to pollen, including pollen from gymnosperms, dicotyledonous angiosperms and monocotyledonous angiosperms, dust mite antigens and mold antigens such as Alternaria antigens.

An unrelated allergen as used here is an allergen that is not significantly bound by the anti-allergen binding domain used in the treatment. Preferably, the binding is less than 10%, more preferably less than 1% of the binding of the specific allergen towards which the anti-allergen binding domain is directed, as measured in an elisa assay.

An allergic inflammation as used here is an airway response characterized by increased infiltration in the airways of leucocytes and/of which eosinophils are the main component. In addition, an increased mucus formation as the result of goblet cell metaplasia characterizes allergic airway inflammation.

### EXAMPLES

### Materials and Methods to the Examples

Mice

Female BALB/c mice were purchased from the Broekman Institute. All mice used were 5-8 wks old at the time of the experiments.

Sensitization and Challenge

The mice were primed intraperitoneally with 10 μg of ovalbumin (OA) (Sigma Chemical Co., St. Louis, Mo.), adsorbed to 1 mg of alum in 0.5 ml of endotoxin free phosphate-buffered saline (PBS), three times at 1-wk intervals. At 7 and 8 days after the last immunization the mice received an aerosol challenge with OA (1%, w/v). This was accomplished in a closed plastic aerosol chamber in which the mice were placed for 30 min. The aerosol was generated via a pressurized nebulizer. Mice were sacrificed 48 hours after aerosol exposure (short protocol). To assess the persistence of the protective effect after multiple aerosol exposures, another protocol involving sensitization of mice with 10 μg OA and 1 mg alum intraperitoneally on days 0 and 7 followed by OA aerosol challenge from days 14 to 20 was used (repetitive protocol). When catalase (human erythrocyte Cat No 219008, Calbiochem) was used as the model allergen, sensitization of mice involved 100 μg h-cat in 2 mg alum and challenge was performed with 50 μg h-cat in 80 μl intratracheally (since catalase is degraded by aerosol).

Generation of Anti-OA Antibody Secreting Hybridoma Cell Lines

Briefly spleen cells (1×10⁸/25 ml) from BALB/c mice immunized with OA were fused with the myeloma cell line SP2/0 (2×10⁷/25 ml) in a 50 ml tube, centrifuged, supernatant discarded and pellet dried at 37° C. Polyethylene glycol was added slowly for 2 min followed by addition of 30 ml serum free medium (RPMI, glutamine, sodium pyruvate and β-mercaptoethanol) over a period of 15 mins. Centrifuge and resuspend the pellet at a concentration of 106 per ml in RPMI medium without HAT. After 24 hrs, add 50 μl of HAT per well. After 10-14 days screen the 96 well plates by ELISA for anti OA IgE and IgG antibodies. Hybridoma cells secreting anti-OA antibodies were recloned several times by limiting dilution. OA-specific subclones from plates were selected, transferred to 24 well plate and cultured for 7-10 days, supernatants were harvested and pooled. The antibodies were later purified by protein G (Pharmacia) affinity column under endotoxin free conditions and the amount of protein determined by ELISA. The antibody was filter sterilized through a 0.22-μm filter before use.

Antibody Treatment

Anti-OA IgG2a antibody (50 μg) was given to mice intranasally 2 hours prior to challenge with aerosol. Preliminary experiments have demonstrated the concentration of 50 μg administered twice to be the optimal dose for suppressing airway allergic inflammation. Further kinetics of retention of intranasally administered anti-OA IgG2a antibody in lungs have indicated the optimal time to be 2-12 hrs. The routes of administration of antibody viz. intranasal vs intravenous for elucidation of protective effect were compared. Intranasal route of administration of antibody markedly reduced the airway eosinophilia as compared to intravenous route, which was not effective.

Analysis of Bronchoalveolar Lavage (BAL) Fluid

The lungs of BALB/c mice were lavaged thrice with 1 ml HBSS (Ca²⁺ and Mg free HBSS supplemented with 0.05 mM sodium EDTA). BAL fluid was centrifuged (10 min, 4° C., 700×g) and the supernatant was stored at −70° C. for analysis of cytokine content. After resuspension in HBSS cells were counted on a haemocytometer. BAL cell differentials were determined on cytospin preparations stained with May Grunwald Giemsa. At least 200 cells were differentiated by light microscopy based on conventional morphologic criteria.

Determination of Cytokine Responses

Intracellular cytokine staining was performed on BAL cells (1×10⁶ cells/ml) which were stimulated with 5 μg/ml anti-CD3 mAb, 5 μg/ml of anti-CD28 and 10 μg/ml brefeldin A (Sigma) for 4 hours at 37° C. in 96 well plates according to the protocol. Control cultures were treated only with anti-CD28 mAb. Brefeldin A was not added to the cultures when supernatants were harvested and cytokine measurements were done by ELISA. At the end of the stimulation cells were stained for three-color flow cytometry. Briefly, cold PBS (Ca²⁺, Mg²⁺ free supplemented with 1% BSA and 0.02% sodium azide (staining buffer) was added to the plates and cells pelleted. The supernatant was discarded and cells incubated with prewarmed 0.02% EDTA in staining buffer for 10 min at 37° C. and vortexed to remove adherent cells. The cells were washed and fixed in 2% formaldehyde. The cells were again washed and resuspended in staining buffer. Next day, the cells were washed and incubated with blocking anti-Fc RII/III mAb 2.4G2 in permeabilization buffer (0.2% saponin in staining buffer) to prevent nonspecific binding followed by surface staining with anti-CD4 cychrome, and anti-IL-4 PE, anti-IL-5 FITC or anti-CD4 cychrome and anti-IFN-γ PE, anti-IL-2 FITC. The above antibodies were obtained from Pharmingen. All incubations were performed on ice. The cells were also pre-incubated with purified blocking anti-cytokine antibodies to evaluate non-specific staining.

The levels of IL-4, IL-5 and IFN-γ protein in the culture supernatants stimulated with anti-CD3 and anti-CD28 as well as in the supernatants of BAL fluid were determined by commercial ELISA kits from Endogen according to the manufacturer's protocol.

Generation of F(ab′)2 Fragments

50% slurry of immobilized pepsin (180 μl) was added to 16×150 mm glass test tube (90 μl gel) followed by addition of 2 ml of digestion buffer (20 mM sodium acetate buffer pH 4.5). The tube was centrifuged to separate the pepsin from buffer. The buffer was discarded and the wash procedure was repeated with another 2 ml buffer. Immobilized pepsin was then resuspended in 0.36 ml of digestion buffer. The anti-OA IgG2a antibody in solution (6 mg/ml) was dialyzed against 200 ml digestion buffer for 4 hrs at 4° C. This was then concentrated to 6 mg/360 μl in centricon tubes molecular weight cutoff 3500 and 0.36 ml of this sample solution was added to 0.36 ml of digestion buffer. This volume of 0.72 ml of sample was added to the tube containing immobilized pepsin and incubated for 6 hrs in a high-speed shaker water bath at 37° C. at high speed. Constant mixing of the gel was maintained during incubation. Then 1.08 ml of IgG binding buffer (10 mM Tris-HCl+0.1% disodium EDTA, pH 8.0) was added to the crude digest in eppendorf tubes. The sample was then run on SDS-PAGE and developed by silver staining. Two bands corresponding to 110-120 Kda and 50 Kda were observed under non-reducing conditions. However, the band corresponding to 150-250 Kda for the undigested antibody was not observed indicating that digestion was complete.

F(ab′)2 Purification

Protein A column was equilibrated with 12 ml binding buffer or more. The crude digest was then layered on to the column and allowed to completely flow into the gel. The column was washed with binding buffer and eluate collected which contains F(ab′)₂ fragments contaminated with small Fc fragments which can no longer bind to Protein A. The eluate was collected in LPS free glass test tubes. The fragments corresponding to F(ab′)2 were run on SDS-PAGE and silver staining performed. F(ab′)2 sample was dialyzed against PBS pH 7.4 before use.

ELISA to Quantitate Protein in F(ab′)₂

Ninety-six -well plate were coated with OA 100 μg/ml in carbonate-bicarbonate buffer overnight. After washing the plates they were blocked with 1% BSA in PBS-0.05% Tween for 2 hrs at 37° C. The plates were again washed and the sample of F(ab′)2 and whole Ab at 1/100 were added and incubated for 2 hrs at RT. Goat anti-mouse kappa (1:1000) (Southern Biotechnology Associates 1 mg/ml) was added and incubated for 1 hr at RT followed by incubation with rabbit anti-goat IgG AP Sigma (1:9000) for 1 hr at RT. Substrate p-nitrophenyl phosphate, disodium was added and plates incubated for 30 mins. The plates were read on an ELISA reader at 405 nm. The concentration of the whole Ab being known, from the titers the concentration of functional F(ab′)₂ was calculated.

Cross Protection Experiment

Mice were sensitized with 10 μg OA and 100 μg h-cat in 2 mg alum on days 0, 7 and 14. Intratracheal challenge was performed with 10 μg OA and 50 μg h-cat on days 21 and 22 (Challenge 1). A second intratracheal challenge with 10 μg OA or 50 μg h-cat was given on day 27 (Challenge 2). A control group administered PBS during challenge 2 was also included. Anti-OA IgG2a and anti-cat IgG2a were administered intranasally two hours before challenge 1 but not before challenge 2. Thus, mice treated with anti-hcat IgG2a and challenged with h-cat and OA, were rechallenged with either h-cat or OA without further treatment with the antibody. Mice treated with anti-OA IgG2a and challenged with h-cat and OA, were rechallenged with OA or h-cat. On day 29, mice were sacrificed, BAL performed and cells counts determined on cytospin preparations.

Histological Analysis

After bronchoalveolar lavage, the lungs were fixed in 4% paraformaldehyde and embedded in paraffin. Sections of 2.5 μm thickness from all lobes were stained either with Congo-Red (demonstrating eosinophils) or Periodic Acid Shiff (demonstrating goblet cells). Slides were coded and the peribronchial (and perivascular) inflammation was graded in a blinded fashion using a reproducible scoring system. Briefly, a value from 0 to 3 was adjudged to each tissue section scored. A value of 0 was adjudged when no inflammation was detectable, a value of 1 for occasional cuffing with inflammatory cells, a value of 2 when most bronchi were surrounded by a thin layer (1 to 5 cells) of inflammatory cells and a value of 3 when most bronchi were surrounded by a thick layer (>5 cells) of inflammatory cells. As 5-7 tissue sections per mouse were scored, inflammation scores could be expressed as a mean value per animal and could be compared between groups. For estimating the presence of mucus producing cells, we counted the number of airways per section and adjudged a score of 0, 1, 2 or 3 to each airway when no, very few, less than 50% or over 50% of the airway epithelial cells were PAS positive. Each mouse and group became as such characterized by a distribution of scores, which could be compared statistically.

Statistical Analysis

All results are expressed as means±standard error of the mean (SEM). Data were analyzed by a non-parametric Mann-Whitney U test.

### Example 1

### Anti-Allergen Antibodies Inhibit Allergen Induced Eosinophilia

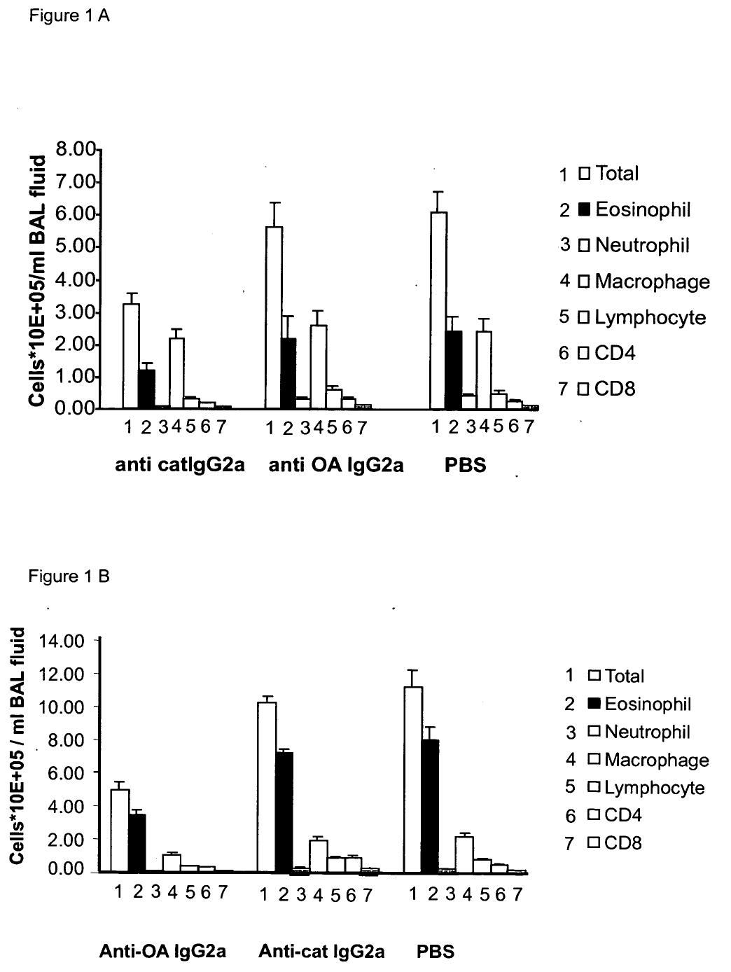

We sensitized mice against two model allergens, human catalase (h-cat) and the commonly used ovalbumin (OA), by repeated intraperitoneal injection of the antigens adsorbed to alum. Subsequent exposure of the sensitized mice to the corresponding nebulized antigen for two consecutive days resulted in a 10-fold (h-cat) to 20-fold (OA) increase in the total bronchoalveolar lavage (BAL) cell count as compared to naive mice. For both model allergens, eosinophils formed the predominant fraction of the cellular infiltrate whereas in h-cat challenged mice a significant recruitment of macrophages was also observed (FIGS. 1A & 1B). The Th2 nature of the airway inflammatory response to both antigens is further exemplified by the mere absence of neutrophils in the BAL. To verify whether anti-allergen antibodies inhibit allergen-induced eosinophilia, we used monoclonal antibodies against the corresponding antigens. We administered 50 μg anti-catalase IgG2a or anti-OA IgG2a intranasally to h-cat or OA sensitized mice respectively, prior to challenge with the corresponding antigen. Experiments involving kinetic studies with anti-catalase antibody demonstrated 80% retention of the antibody in the lungs after 2-3 hours followed by a sharp decline after 24 hours. Therefore in the present study, we administered anti-allergen IgG2a two hrs prior to intratracheal/aerosol challenge with either antigen. Cellular infiltration in BAL was determined after treatment with the antibody and subsequent airway challenge to assess the allergen-specific interference by the antibody with the allergic airway inflammation. Treatment with allergen matched monoclonal antibody demonstrates that anti-allergen IgG2a accompanied by subsequent intratracheal/aerosol challenge led to a 50% reduction in the BAL cell count with either model allergen compared to the control (placebo) group which received PBS instead of the antibody (FIGS. 1A & 1B). The cellular infiltrate of the antibody treated group also showed a substantial decrease in the amount of eosinophils as compared to the placebo group. In contrast, the amount of macrophages, neutrophils and lymphocytes were not altered by treatment with allergen-specific IgG2a.

To assess whether formation of antigen: antibody complexes is necessary for the protective effect of the administered antibodies, we treated the sensitized mice with mismatched antibodies, and challenged them 2 hours later with the correct antigen. Treatment of h-cat sensitized and challenged mice with anti-OA IgG2a did not alter the size and composition of the bronchoalveolar cell infiltrate when compared with the placebo-treated group (FIG. 1A). Also in the opposite experiment, namely administration of anti-hcat IgG2a to OA-sensitized mice followed by OA challenge, no effect on the BAL cell count and composition were observed (FIG. 1B). From these results we conclude that anti-allergen IgG2a, administered to the upper airways by intranasal instillation protects sensitized mice from allergen induced airway eosinophilia provided antibody specificity and allergen are matched.

### Example 2

### Anti-OA IgG2a Inhibits Airway Eosinophilia Induced by Repetitive OA challenge

In order to examine the effect of prolongation of the aeroallergen challenge in OA sensitized mice, we exposed the mice to seven consecutive OA aerosols and treated the mice with 50 μg anti-OA IgG2a or PBS as placebo on days 0 and 3 (as described in Materials and Methods). We also included a third group of mice treated with 100 μg anti-OA IgG2a on day 0. We observed that 50μg of anti-OA IgG2a followed by repetitive allergen exposure reduced the cellular infiltration in the lung by 50% as compared to the placebo group (FIG. 1C). Treatment with 100 μg antibody also inhibited the airway inflammation by 21% relative to the placebo group indicating that a single administration of the antibody is not as effective as the antibody administered twice. This experiment indicates that the inhibition of allergic airway inflammation by anti-allergen antibodies is not a passive mechanism as a consequence of immune shielding but instead involves an active modulation of the immune response due to the remarkable persistence of inflammation repression even after exposure to seven aerosols.

We also ascertained whether the suppression of airway eosinophilia is isotype specific (IgG2a specific). In this regard, we sensitized and challenged mice with OA by the repetitive protocol (as described in Materials and Methods) and treated with 50 μg of anti-OA IgG2a, anti-OA IgG2b, anti-OA IgG1 or PBS respectively on days 0 & 3. Cellular infiltration in BAL determined 24 hours after the last aerosol exposure demonstrates that all the anti-allergen IgG isotypes were able to suppress airway eosinophilia to a similar extent (FIG. 1D) thereby indicating that all tested IgG isotypes are equally potent in inducing the above protective effect.

### Example 3

### Suppression of Airway Eosinophilia by IgG Treatment is Dependent on Fc-Receptor Mediated Endocytosis

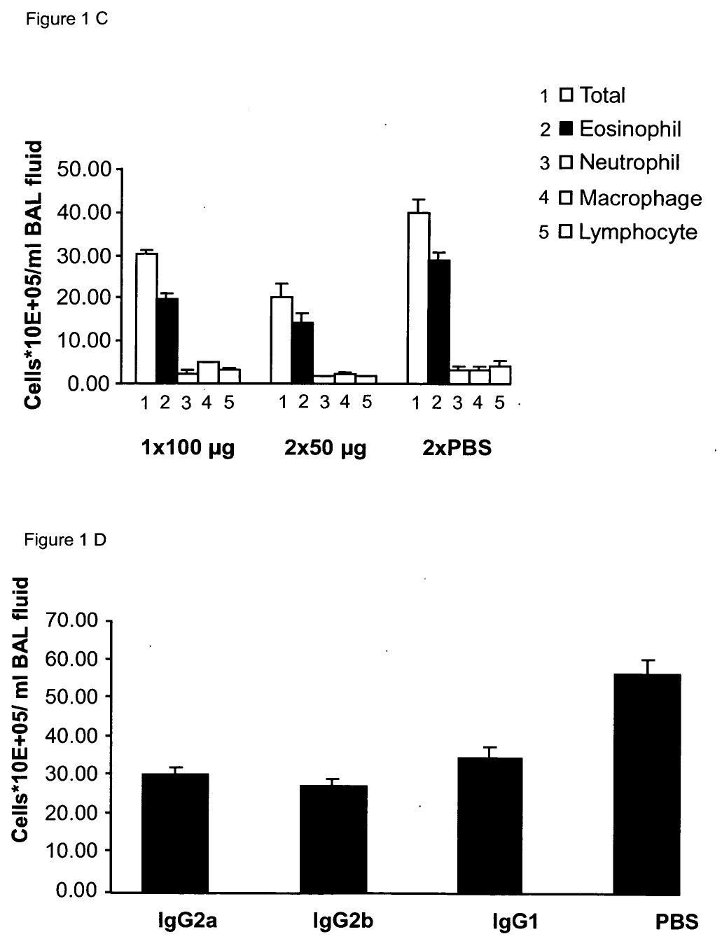

Antibody-antigen complexes are known to trigger various biological responses in effector cells by binding to Fey receptors present on the surfaces of the cells. Because Fcγ receptors specifically interact with the Fc domain of IgG, removal of this domain generates a F(ab′)₂ fragment that is no longer capable of binding to these receptors while retaining the cognate activity of the intact antibody. To investigate whether this interaction of antibody-antigen complexes with Fcγ receptors is part of the mechanism by which anti-allergen IgG suppresses eosinophilic airway inflammation, equimolar concentrations of functionally active F(ab′)₂ fragments of anti-OA IgG2a (generated by pepsin cleavage as described in materials and methods) and of native IgG2a were administered to OA sensitized mice two hours prior to aeroallergen challenge. The challenge consisted of seven consecutive exposures to OA aerosols. The results demonstrate that anti-OA IgG2a significantly reduces the airway inflammatory response by 70% whereas F(ab′)₂ fragments derived from the same antibody failed to reduce the airway inflammatory response (FIG. 2). The above experiment reveals that the IgG-Fcγ receptor interaction is critical for the anti-inflammatory action exerted by topical anti-allergen IgG.

### Example 4

### Anti-OA IgG, but not F(ab′)₂ Represses Cell Infiltration in the Lungs and Goblet Cell Metaplasia

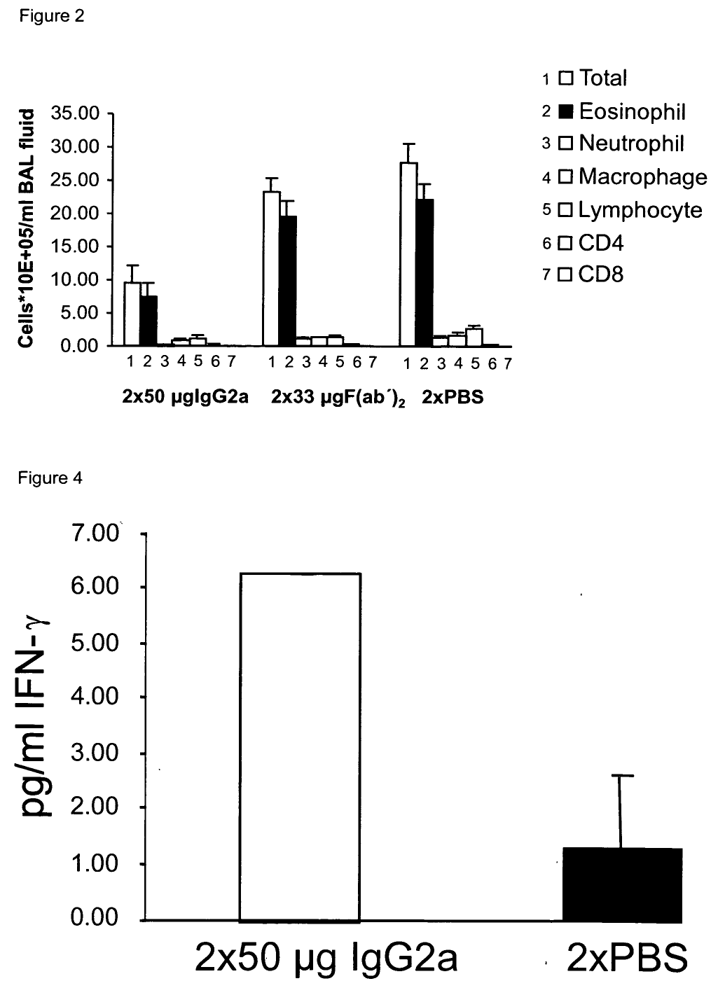

To further characterize in the different groups the inflammatory responses ongoing within the lung tissue, lung sections were stained with Congo-Red (demonstrating eosinophils) and scored for magnitude of peribronchial and perivascular cell infiltration. Histological analysis of the lungs from the placebo-treated mice exposed to the nebulized allergen, showed infiltration of the airways as well as the perivascular areas with eosinophils and mononuclear cells (FIG. 3, upper panels). Alveolar septa were not infiltrated with inflammatory cells. Treatment of these allergen-exposed mice with anti-OA-F(ab′)₂ did not alter the histology of the airways. In contrast, treatment with anti-OA-IgG2a prior to and during the allergen challenge reduced the airway inflammation significantly as compared to the placebo or F(ab′)₂ treated mice (FIG. 3, upper panels). This difference was also seen in the perivascular areas, though no statistical significance was reached here. Specific staining for mucus producing cells (PAS) revealed in the placebo-treated animals that 32±8% of the airways were free of PAS-positive cells (FIG. 3, lower panels). This FIG. is similar to that of the mice treated with F(ab′)₂: 29±4%, p>0.05. However, much more airways were free of PAS-positive cells in the mice treated with anti-OA-IgG2a: 49±4%; p<0.05. While no significant differences were found for the airways with few or moderate numbers of mucus cells, there were significant differences in the presence of airways of which more than 50% of the epithelial cells were PAS-positive: 53±6% for placebo treated animals versus only 32±4% for anti-OA-IgG2a treated animals (p<0.05). From these results an anti-inflammatory activity can be assigned to the presence of IgG in the lumen of the airways, requiring Fc domain-mediated effector functions and featuring a reduced eosinophil influx in the airways and lung perivascular areas, along with a reduction of mucous cell metaplasia.

### Example 5

### Anti-OA IgG2a Increases the Participation of Th1 Cells to the Anti-Allergen Immune Response

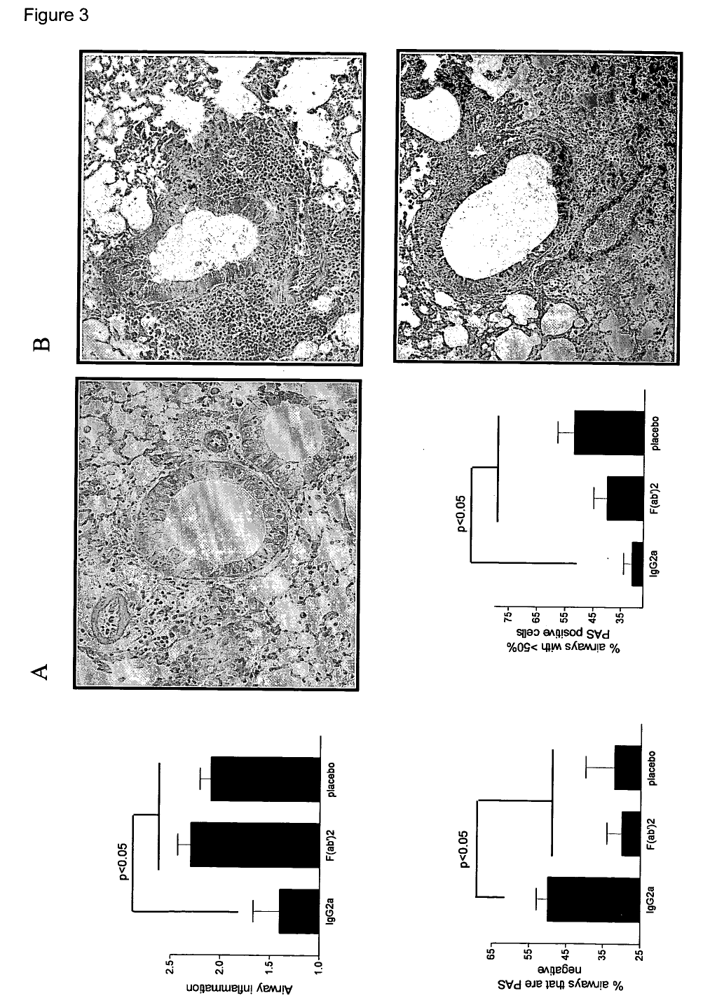

Interference of the anti-allergen IgG treatment with T cell responses in the airways was verified on the basis of changes in IL-4, IL-5 and IFN-γ levels present in the BAL fluid. Mice assayed for this purpose were exposed to seven consecutive OA aerosols and treated with anti-OA IgG2a or PBS as placebo on days 0 and 3 (as described in Materials and Methods). Strikingly, a marked increase in the level of the Th1 cytokine IFN-γ was observed in the bronchoalveolar fluid of mice treated with anti-OA IgG2a (FIG. 4), whereas the levels of the Th2 cytokines IL-4 and IL-5 were not significantly altered. To verify whether this IFN-γ increment reflects an increased participation of Th1 cells to the allergen-induced airway immune response, the relative numbers of IFN-γ, IL-4 or IL-5 secreting CD4⁺ T cells present in the BAL were determined by intracellular cytokine staining. T cells were optimally stimulated by addition of anti-CD3 and anti-CD28 antibody cocktail, thus providing both signal 1 and signal 2 to the T cells. As a negative control, cells that only received signal 2 (anti-CD28 antibody) were also included. Four hours after stimulation, the cells were stained with anti-CD4 and anti-cytokine antibodies and analyzed on a flow cytometer. Our results reflect that treatment with anti-OA IgG2a doubled the frequency of IFN-y secreting CD4⁺ T cells concomitant with a decline in the frequency of IL-4 secreting CD4⁺ T cells, thus resulting in a three fold increase of the ratio of Th1 to Th2 cells in the BAL of antibody treated mice (Table 1). The frequency of IL-5 secreting CD4⁺ T cells remained unaltered upon treatment with the antibody. From these results we conclude that administration of anti-allergen antibody to the upper airways followed by exposure of the airways to the specific allergen increases the participation of Th1 cells to the immune response and thereby may counteract the Th2-driven eosinophilic inflammatory response of the airways.

### Example 6

### Protection Persistence of Anti-Allergen Antibodies on Re-Exposure to Allergen and Cross Protection Against Unrelated Bystander Allergen

The cytokine environment at the time of antigen challenge is crucial in determining the type of immune response that will be induced. Accordingly, the pro-Th2 environment of the airways is considered as an important factor in the development of airway hyperreactivity to inhaled allergens. Thus, asthmatic individuals may mount allergic responses to secondary bystander allergens due to their prevailing Th2 environment. We therefore verified whether an increased participation of Th1 cells induced by treatment with anti-allergen IgG2a would also promote Th1 responses against unrelated allergens, thus providing cross protection against bystander allergens. To test the above hypothesis, mice were rendered sensitive simultaneously to two allergens namely OA and h-cat. Occurrence of protection persistence and cross protection were analyzed by intranasal administration of one of the allergen matched IgG2a antibodies to dual sensitized mice during the first round of allergen challenge followed five days later by a second round of allergen challenge. However, during the second challenge the mice were challenged with a single allergen, either the correct or the mismatched allergen with respect to the specificity of the antibody instilled during the first round of allergen challenge. Thus, mice treated with anti-hcat IgG2a and challenged with h-cat and OA, were re-challenged with either h-cat or OA without further treatment with the antibody. Inversely, mice treated with anti-OA IgG2a and challenged with h-cat and OA, were re-challenged with OA or h-cat. In both cases, a clear reduction of the BAL cell infiltration and airway eosinophilia were observed, despite the mismatch between the treating antibody given during the first challenge and the allergen instilled during the second challenge (Table 2). From this experiment we conclude that the protective effect of the antibody persists during a second round of allergen challenge. Furthermore, the interaction of the antibody with its specific allergen also offers protection against allergic airway inflammation generated by a concomitantly present secondary allergen. Therefore, topical application of a single anti-allergen IgG provides cross-protection against unrelated bystander allergens.

TABLE 1
Percentage cytokine-positive CD₄⁺ T cells

| | Placebo | 2 × 50 μg Ab |
|---|---|---|
| IFN-γ | 3.0 | 6.1 |
| IL-4 | 12.3 | 8.5 |
| IFN-γ/IL-4 | 0.24 | 0.71 |

Intracellular cytokine staining of CD4 lymphocytes in BAL. Mice were treated according to the protocol of repetitive OA aerosol challenge and BAL cells from each group were pooled and stimulated in vitro with anti-CD3 & anti-CD28 antibodies for 4 hrs in the presence of Brefeldin A.

TABLE 2

| | % Protection against 2^{nd} challenge with | | |
|---|---|---|---|
| Treatment | PBS | h-cat | OA |
| Anti-hcat IgG | 98 (95) | 60 (79) | 64 (56) |
| Anti-OA IgG | 98 (97) | 48 (47) | 61 (66) |
| PBS | 100 (100) | 0 (0) | 0 (0) |

Anti-allergen IgG2a exerts protection against specific and bystander aeroallergen airway inflammation. We sensitized mice with h-cat and OA simultaneously followed by intratracheal challenge with hcat and OA (Challenge 1). Mice received a second intratracheal challenge with hcat, OA or PBS five days after the first round of allergen challenge (Challenge 2). Anti-cat IgG2a, anti-OA IgG2a or PBS were administered to the mice two hours during the first challenge with OA and hcat (Challenge 1). 48 hrs after the second round of allergen challenge, we determined the total BAL cell count and cellular infiltration in BAL. Data are expressed in terms of the degree of protection (%) conferred by the respective antibodies. Values refer to total cell counts, the values in parentheses represent the values for eosinophil counts.

### References

Azuma, I., K. Sugimora, T. Taniyama, M. Yawakawi, Y. Yamamura, S. Kusumoto, S. Okada, and T. Shiba. 1976. Adjuvant activity of mycobacterial fractions: hnmunological properties of synthetic N-acetylmuramyl dipeptide and related compounds. Infect. Immun. 14:18-27.
Corry, D. B., and F. Kheradmand. 1999. Induction and regulation of the IgE response. Nature 402-supp: B18.
de Jong, R., A. A. Janson, W. R. Faber, B. Naafs and T. H. Ottenhoff. 1997. IL-2 and IL-12 act in synergy to overcome antigen-specific T cell unresponsiveness in mycobacterial disease. J. Immunol. 159:786-793.
Donckier, V., D. Abramowicz, C. Bruyns, S. Florquin, M. L. Vanderhaeghen, Z. Amraoui, C. Dubois, P. Vandenabeele, and M. Goldman. 1994. IFN-gamma prevents Th2 cell-mediated pathology after neonatal injection of semiallogenic spleen cells in mice. J. Immunol. 153:2361-2368.
Galli, S. J. 2000. Allergy. Current Biology 10:R93.
Heinzel, F. P., M. D. Sadick, S. S. Mutha, and R. M. Locksley. 1991. Production of IFN-γ, IL-2, IL-4 and IL-10 by CD4⁺ lymphocytes in vivo during healing and progressive murine leishmaniasis. Proc. Natl. Acad. Sci. USA 88:7011- 7015.
Holt, P. G., C. Macaubas, P. A. Stumbles, and P. D. Sly. 1999. The role of allergy in the development of asthma. Nature 402-supp: B12.
Leonard, J. P., K. E. Waldburger, and S. J. Goldman. 1995. Prevention of experimental autoinmune encephalomyelitis by antibodies against interleukin 12. J. Exp. Med. 181:381-386.
Mosmann, T. R., and R. C. Coffman. 1989. Th1 and Th2 cells: different patterns of lymphokine secretion lead to different functional properties. Annu. Rev. Immunol. 7:145-173.
Murphy, J. W., B. A. Wu-Hsieh, L. M. Singer-Vermes, A. Ferrante, S. Moser, M. Ruso, S. A. Vaz, E. Burger, V. L. Calich, and I. C. Kowanko. 1994. Cytokines in the host response to mycotic agents. J. Med. Vet. Mycol. 32 suppl 1:203-210.
Nabors, G. S., L. C. C. Afonso, J. P. Farrell, and P. Scott. 1995. Switch form a type 2 to a type 1 T helper cell response and cure of established Leishmania major infection in mice is induced by combination therapy with interleukin 12 and pentostam. Proc. Natl. Acad. Sci. USA 92:3142-3146.
Racke, M. K., A. Bonomo, D. E. Scott, B. Cannella, A. Levine, C. S. Raine, E. M. Shevach, and M. Rocken. 1994. Cytokine-induced immune deviation as a therapy for inflammatory autoimmune disease. J. Exp. Med. 180:1961-1966.
Racke, M. K., D. Burnett, S. H. Pak, P. S. Albert, B. Cannella, C. S. Raine, D. E. McFarlin, and D. E. Scott. 1995. Retinoid treatment of experimental allergic encephalomyelitis. IL4 production correlates with improved disease course. J. Immunol. 154:450-458.
Roussel, E., M. C. Gingras, E. A. Grimm, J. M. Bruner, and R. P. Moser. 1996. Predominance of a type 2 intratumoral immune response in fresh tumor-infiltrating lymphocytes from human gliomas. Clin. Exp. Immunol. 105:344-352.
Vogel, G. 1997. New clues to asthma therapies. Science 276:1643-1646.
Wills-Karp, M. 1999. Immunologic basis of antigen-induced airway hyperresponsiveness. Annu. Rev. Immunol. 17:255-281.
Yamamura, M., K. Uyemura, and R. J. Deans. 1991. Defining protective responses to pathogens: cytokine profiles in leprosy lesions. Science 254:277-279.
Yamauchi, N., S. Matunobo, K. Ito, and T. Miyamoto. 1983. Experimental allergic asthma in guinea-pigs induced by the passive transfer of antibodies to sensitized cells. Jap. J. Allergology 32, 1084-1092.
Yang, X., S. Wang, Y. Fan, X. Han, and J. Yang. 2000. Mycobacterium bovis BCG infection inhibits established allergic reaction in murine asthma-like model. FASEB J. 14, A1065, 100.2.

1. A method for obtaining cross-protection against one or more bystander allergens, said method comprising:
administering a compound comprising an allergen binding domain and a Fc domain, wherein said bystander allergens are unrelated to the allergen bound by said allergen binding domain.

2. The method according to claim 1, wherein said compound is an antibody.

3. The method according to claim 2, wherein said antibody is an IgG isotype antibody.

4. The method according to claim 1, wherein said administration is intranasal or by inhalation.

5. A pharmaceutical composition for the treatment of an allergic disease, said pharmaceutical composition comprising:
at least one compound comprising an allergen binding domain or allergen binding domains and a Fc domain,
wherein said allergic disease is caused by at least one allergen other than the allergen or allergens bound by the allergen binding domain or allergen binding domains.

6. The pharmaceutical composition of claim 5, wherein said compound is an antibody.

7. The pharmaceutical composition of claim 6, wherein said antibody is an IgG isotype antibody.

8. The pharmaceutical composition of claim 5, wherein said allergic disease is allergic asthma.

9. The pharmaceutical composition of claim 5, wherein said allergic disease is allergic rhinitis.

10. The pharmaceutical composition of claim 6, wherein said allergic disease is allergic asthma.

11. The pharmaceutical composition of claim 6, wherein said allergic disease is allergic rhinitis.

12. The pharmaceutical composition of claim 7, wherein said allergic disease is allergic asthma.

13. The pharmaceutical composition claim 7, wherein said allergic disease is allergic rhinitis.

14. The method according to claim 2, wherein said administration is intranasal or by inhalation.

15. The method according to claim 3, wherein said administration is intranasal or by inhalation.
